(19) United States
(12) Patent Application Publication
(10) Pub. No.: US 2023/0086345 A1
(43) Pub. Date: Mar. 23, 2023

(54) NOVEL METHOD OF ENHANCED DRUG DELIVERY TO THE NERVOUS SYSTEM

(71) Applicant: The General Hospital Corporation, Boston, MA (US)

(72) Inventors: Richard Rox Anderson, Boston, MA (US); Lilit Garibyan, Newton, MA (US); Ying Wang, Winchester, MA (US)

(73) Assignee: The General Hospital Corporation, Boston, MA (US)

(21) Appl. No.: 17/794,934

(22) PCT Filed: Jan. 22, 2021
(86) PCT No.: PCT/US2021/014789

§ 371 Date: Jul. 22, 2022

Related U.S. Application Data
(60) Provisional application No. 62/964,988, filed on Jan. 23, 2020.

(51) Int. Cl. A61K 33/00; A61K 9/00

(52) U.S. Cl. CPC A61K 33/00; A61K 9/0019

(57) ABSTRACT

Disclosed herein are methods that allow for efficient delivery of one or more agents to one or more peripheral nerves or the central nervous system by reversibly breaking down one or more of the perineurial (e.g., nerve-tissue), endoneurial (e.g., blood-nerve or “BNB”), Schwann cell, and blood-brain barriers.

The present application claims priority under 35 U.S.C. § 119(e) to U.S. Provisional Patent Application No. 62/964,988, filed Jan. 23, 2020, the contents of which are hereby incorporated by reference in their entirety.

### BACKGROUND OF THE INVENTION

Peripheral nerves are wrapped with protective layers of connective tissue composed of epineurium, perineurium, and endoneurium. This in turn is wrapped with layers of myelin sheaths. These physical barriers function to shield the impulse-conducting elements. Similarly, portions of the central nervous system are surrounded by a diffusion barrier known as the blood-brain barrier (BBB), which is composed of endothelial cells, astrocyte end-feet, and pericytes. See Ballabh, P. et al. Neurobiol. Dis. 2004, 16(1), 1-13. Tight junctions, present between the cerebral endothelial cells, form a diffusion barrier, which selectively excludes most blood-borne substances from entering the brain. Id. Astrocytic end-feet tightly ensheath the vessel wall and appear to be critical for the induction and maintenance of the tight junction barrier, but astrocytes are not believed to have a barrier function in the mammalian brain. Id.

These barriers also establish a stable environment and prevent penetration of harmful agents to the nerve and brain. However, they also limit the penetration of drugs developed to target the nerve or brain for therapy. Thus, administering agents such as drugs, compounds, nucleic acids (such as those in mRNA, RNAi, and viral vector-based drugs), and biologics, to a peripheral nerve to block or relieve neuropathic pain depends on the ability of the agent to diffuse across the endoneurium, perineurium and/or blood-nerve barrier (“BNB”). For example, local anesthetics or any drugs developed for targeting peripheral nerves, must penetrate the epineurium, perineurium, and endoneurium in order to reach their site of target. Thus, higher concentrations of local anesthetics are used clinically than in isolated nerves. At higher concentrations, most local, systemic, or new anesthetics have systemic toxicities, limiting their use in clinics. In addition, certain agents such as biologics have not been used successfully to treat peripheral nerves due to their inability to cross the peripheral nerve barriers to reach the site of action. Oral or intravenous pain medications also have difficulty crossing the blood-nerve barrier to reach the target site of action in the nerve. Similar considerations apply to the administration of agents such as drugs, compounds, nucleic acids, and biologics to the brain to treat the central nervous system.

There is accordingly an unmet need for ways to improve the ability for small molecules, biologics, nucleic acid-based drugs, or anesthetics to flux across the biological barrier surrounding peripheral nerves and the brain.

### SUMMARY OF THE INVENTION

Disclosed herein are methods that allow for efficient delivery of one or more agents to one or more peripheral nerves by temporarily, rapidly, and reversibly breaking down one or more of the perineurial (e.g., nerve-tissue), endoneurial (e.g., BNB), and Schwann cell barriers. These methods allow for more efficient and effective drug delivery to the site of action within the peripheral nerve by improving and facilitating the delivery and diffusion of agents across the intact perineurial, endoneurial, and/or Schwann cell barriers. These methods allow for more efficient and effective drug delivery to the site of action within the peripheral nerve by improving and facilitating the delivery and diffusion of agents across the intact perineurial, endoneurial, and/or Schwann cell barriers. Also disclosed herein are methods that allow for efficient delivery of one or more agents to the central nervous system by reversibly breaking down one or more of endothelial cell, astrocyte end-feet, and pericyte barriers. These methods allow for more efficient and effective drug delivery to the site of action within the central nervous system by improving and facilitating the delivery and diffusion of agents across the intact blood-brain barrier. These methods cause a temporary and transient opening of the perineurial, endoneurial, and Schwann cell barriers. Further, these methods do not cause damage to the nerve endothelial tissue or the surrounding tissue and selectively target the appropriate tissue.

Cold slurries (e.g., ice slurries) are known in the art as compositions that are made of sterile water that forms a plurality of ice particles, excipients or additives such as freezing point depressants in various amounts, and, optionally, one or more active pharmaceutical ingredients, as described in U.S. application Ser. No. 15/505,042 (“'042 application”; Publication No. US2017/0274011), incorporated in its entirety herein. The methods disclosed herein utilize an ice slurry that “primes” a nerve, i.e., opens the barriers around the nerve, for more effective treatment with one or more agents. In some embodiments, the agent is a small molecule, a biologic, a targeted ion channel blocker, an anesthetic, a nucleic acid, an RNA- or DNA-based therapeutic, or a combination thereof.

The improvement in delivery of agents to the peripheral nerves provided by the methods disclosed herein is useful in a broad range of applications, including treatment of neuropathic pain, traumatic nerve injury, inducing anesthesia, nerve block, and/or the treatment of autoimmune diseases that affect the nerves. The methods disclosed herein are also useful for the application of local anesthetics, reducing toxicity by lowering the systemic dose needed to achieve adequate nerve block. The disclosed methods are particularly useful for biologics or other molecules that are unable to cross the blood-nerve barrier. The disclosed methods are also useful for the transport and storage of long-acting drugs at the target nerve site (e.g., liposomal bupivacaine), allowing for the drugs to have a longer duration of action.

The methods disclosed herein help improve the delivery of agents, such as drugs, biologics, growth factor, nucleic acids, or anesthetics, such as tetrodotoxin, bupivacaine, or QX314. Drugs can include any chemical substance that causes a change in a patient's physiology or psychology when consumed. Biologics are products can be composed of sugars, proteins, peptides, antibodies, or nucleic acids or complex combinations of these substances, or may be living entities such as cells and tissues. Biologics can include vaccines, blood and blood components, gene therapy, tissues, recombinant therapeutic proteins, allergenics, and somatic cells. Growth factors are naturally occurring substances capable of stimulating cell proliferation, wound healing, and cellular differentiation. Such growth factors can include nerve growth factor (NGF), brain-derived neurotrophic factor (BNDF), or glial-derived neurotrophic factor (GDNF). Nucleic acid therapeutics are based on nucleic acids or closely related chemical compounds. These include messenger RNAs, small interfering RNAs, antisense oligonucleotides, aptamers, RNA, and DNA-modified gene therapy.

In some embodiments, injected ice slurry is used to reversibly break down one or more of the perineurial (e.g., nerve-tissue), endoneurial (e.g., blood-nerve or “BNB”), Schwann cell, and endothelial cell barriers. In some embodiments, injected ice slurry is used to reversibly break down one or more of the, astrocyte end-feet and pericyte barriers. In some embodiments, injected ice slurry facilitates targeted delivery of agents such as drugs, compounds, and biologics to the peripheral nerve or brain.

In some embodiments, the methods disclosed herein utilize a combination of one or more agents with ice slurry. In some embodiments, the one or more agents are applied with the ice slurry. In some embodiments, the one or more agents are administered after the ice slurry. For example, the agent may be administered about 5 minutes, between about 5 and about 10 minutes, between about 10 minutes and about 1 hour, between about 1 hour and about 6 hours, between about 6 hours and about 12 hours, between about 12 hours and about 18 hours, between about 18 hours and about 24 hours, and between about 24 hours and about 36 hours after administration of the ice slurry. In some embodiments, the one or more agents comprises a local anesthetic. In some embodiments, the combination of one or more agents with ice slurry improves one or more of (1) the duration of effect (e.g., the duration of the nerve block provided by a local anesthetic), (2) the penetration of the one or more agents to the site of action, and (3) the amount of the one or more agents needed for biologic response (e.g., requiring a reduced amount of the one or more agents, and therefore reducing unwanted side effects).

In some embodiments, the ice slurry is injected around the peripheral nerve of a patient that is targeted for treatment. In some embodiments, the ice slurry is injected around the brain of a patient that is targeted for treatment. In some embodiments, the ice slurry is injected around the spinal cord of a patient that is targeted for treatment. In some embodiments, the agent is delivered by intravenous injection, local injection, oral administration, or a combination thereof.

In some embodiments, disclosed herein is a drug-device combination where the drug of interest is mixed with the slurry and then injected to the target site. For example, tetrodotoxin is a very potent nerve blocker that can block nerves for many hours. However, it has high systemic toxicity at doses needed for reducing pain. When delivered with slurry or delivered after slurry treatment of the nerve as disclosed herein, the dose of tetrodotoxin needed for nerve block is reduced, thus reducing and/or preventing systemic toxicity.

### BRIEF DESCRIPTION OF THE DRAWINGS

FIG. 1 includes schematics of the blood-nerve barrier published in Richner, M. et al. Frontiers Neurosci. 2019, 12(1038), 1-9. FIG. 1A depicts a transverse view schematic of a peripheral nerve ensheathed by epineurial collagen fibrils (epineurium) and blood vessels. Individual nerve fascicles consisting of unmyelinated and myelinated axons as well as small blood vessels are ensheathed by the perineurium, forming the endoneurial microenvironment. FIG. 1B depicts a schematic of an individual endoneurial blood vessel surrounded by endothelial cells, pericytes and the basement membrane. FIG. 1C depicts a schematic of the cellular structure of the blood-nerve barrier, formed by endothelial cells, that are connected by tight junctions, pericytes and the basement membrane. The barrier is exposed to cells and molecules circulating in the blood, protecting constituents of the endoneurium (Remak bundles, myelinated axons, resident macrophages and fibroblasts) from toxic factors. FIG. 1D depicts a schematic of endothelial cells, which are tightly interconnected by tight junctions and adherens junctions forming a restrictive intercellular barrier. Zona occludens-1 and -2 (ZO-1, ZO-2) interact with claudin-5, occludin and likely with claudin-12/19 forming tight junctions. β-catenin forms in conjunction with VE-cadherin adherens junctions.

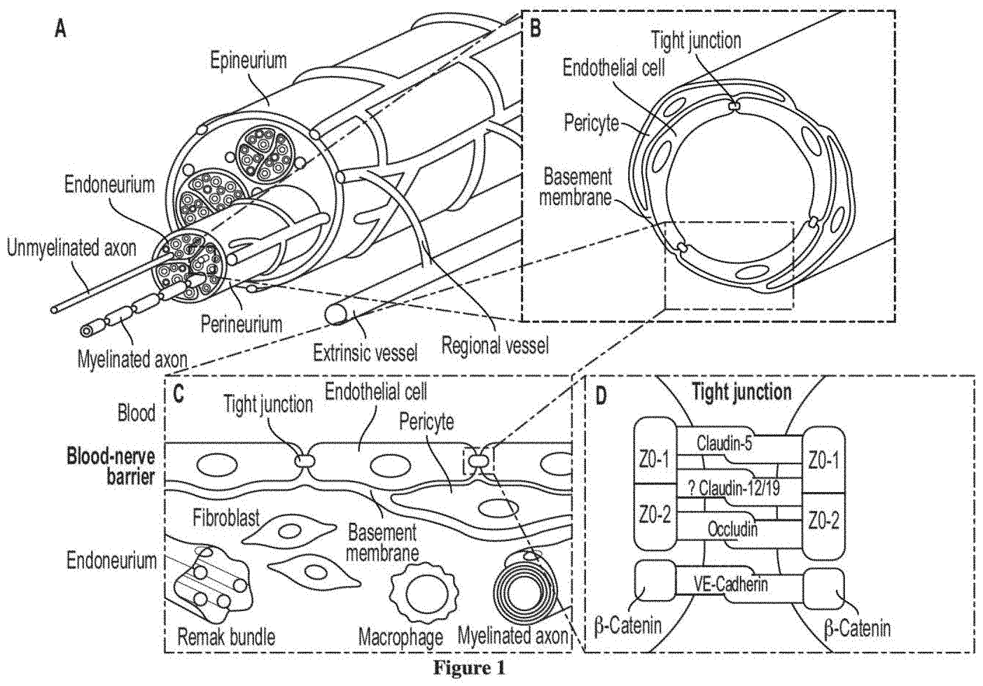

FIG. 2 depicts a schematic of a peripheral nerve adapted from a presentation by Wayan Sugiritama, Educational Staff at Medical Faculty of Udayana University, published Jun. 23, 2009 (available at https://www.slideshare.net/sugiritama/histologic-structure-of-nervous-system, last visited Jan. 21, 2020). FIG. 2 further depicts the epineurium, composed of dense collagenous connective tissue with thick elastic fiber, which prevents damage by overstretching. FIG. 2 also depicts the perineurium, composed of dense connective tissue, layers of epithelioids, and which isolates the neural environment (i.e., the blood-nerve barrier). FIG. 2 further depicts the endoneurium, composed of loose connective tissue which regulates the microenvironment of the nerve fiber.

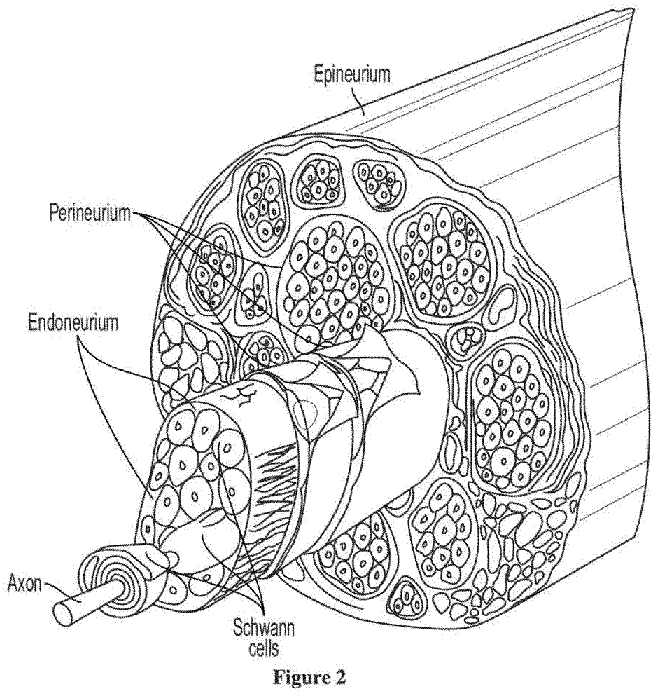

FIG. 3 depicts a schematic of the blood-brain barrier and the tight junction published in Ballabh, P. et al. Neurobiol. Dis. 2004, 16(1), 1-13. FIG. 3A depicts a schematic drawing of the blood-brain barrier in transverse section showing endothelium, basement membrane, pericytes, astrocytes, and tight junctions. The localization of gap junction, GFAP, and aquaporin-4 are shown. FIG. 3B depicts an electron micrograph of mammalian blood-brain barrier showing endothelial tight junction, which was in turn adapted from: The Blood-Brain Barrier Cellular and Molecular Biology Pardridge, W. M. (ed.), Raven Press. FIG. 3C depicts a schematic representation of protein interaction associated with tight junctions at the blood-brain barrier. Claudin, occludin, and junction adhesion molecule are the transmembrane proteins, and ZO-1, ZO-2, and ZO-3, cingulin, and others are the cytoplasmic proteins. Claudins are linked to actins through intermediary cytoplasmic proteins.

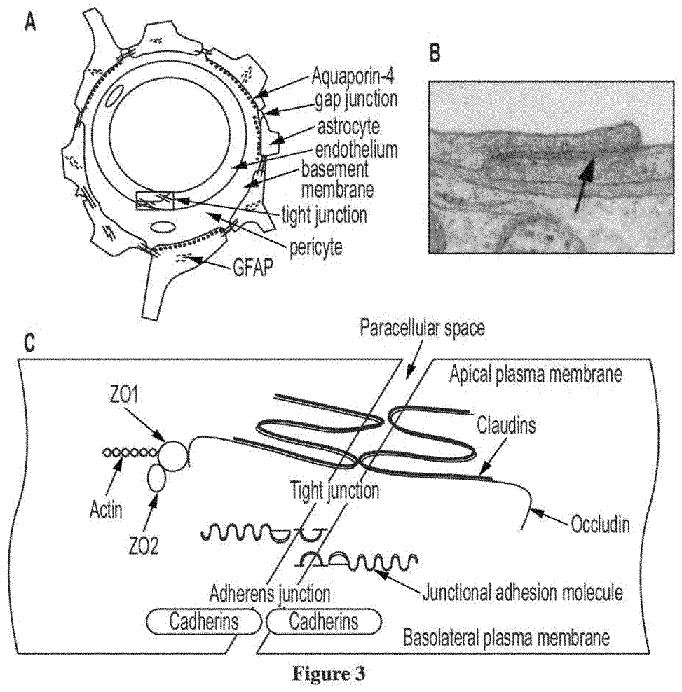

FIGS. 4A-C depict the results of day 1 after ice slurry injection in rats where Evans Blue (“EB”) dye was injected through the vein to test the permeability changes of blood vessels in the endoneurium of the sciatic nerve. FIG. 4A depicts the control side, FIG. 4B depicts the ice slurry treated side, and FIG. 4C depicts the room temperate slurry treated group.

FIGS. 5A-C depict the results of day 3 after ice slurry injection in rats where EB dye was injected through the vein to test the permeability changes of blood vessels in the endoneurium of the sciatic nerve. FIG. 5A depicts the control side, FIG. 5B depicts the ice slurry treated side, and FIG. 5C depicts the room temperate slurry treated group.

FIG. 6 shows the results of the vascular permeability assay after ice slurry treatment at day 1 post treatment.

FIG. 7 shows the results of the vascular permeability assay after ice slurry treatment at day 3 post treatment.

FIG. 8 shows the combined results of the vascular permeability assay after ice slurry treatment.

FIG. 9 shows the combined results of the vascular permeability assay after room temperature slurry treatment.

FIG. 10 shows the combined results of the vascular permeability assay after ice slurry and room temperature slurry treatment.

FIGS. 11A-F show confocal images of the EB dye extravasation from the blood vessels within the endoneurium at day 1. The lighter portions of the image indicate where dye is present.

FIGS. 12A-F show confocal images of the EB dye extravasation from the blood vessels within the endoneurium at day 3. The lighter portions of the image indicate where dye is present.

FIGS. 13A-F show transmission electron microscopy (TEM) images of the sciatic nerve at day 1. The white arrows on FIGS. 13B, 13D, and 13F indicate the location of endothelial tight junctions.

FIGS. 14A-L show confocal images of different types of FITC-Dextran dye extravasation from the blood vessel within the endoneurium at day 1. The lighter portions of the image indicate where dye is present.

FIGS. 15A-B show confocal and bright-field images of the EB dye and FITC-Dextran dye extravasation from the blood vessel within the endoneurium. The lighter portions of the confocal images indicate where dye is present.

FIGS. 16A-L show confocal and bright field images of the FITC-Dextran 70 dye distribution across the perineurium at day 1 where the dye was injected 5 minutes after administering either slurry or the control. The lighter portions of FIGS. 16A, 16C, 16E, 16G, 16I, and 16K indicate where dye is present.

FIGS. 17A-L show confocal and bright-field images of the FITC-Dextran 70 dye distribution across the perineurium at day 1 where the FITC-Dextran 70 dye was injected 1 day after administering either slurry or the control. The lighter portions of FIGS. 17A, 17C, 17E, 17G, 17I, and 17K indicate where dye is present.

FIGS. 18A-L show confocal and bright-field images of the FITC-Dextran 70 dye distribution across the perineurium at day 1 where the dye was injected 5 minutes after administering either slurry or the control. The lighter portions of FIGS. 18A, 18C, 18E, 18G, 18I, and 18K indicate where dye is present.

FIGS. 19A-L show confocal and bright-field images of the FITC-Dextran 70 dye distribution across the perineurium at day 1 where the FITC-Dextran 70 dye was injected 1 day after administering either slurry or the control. The lighter portions of FIGS. 19A, 19C, 19E, 19G, 19I, and 19K indicate where dye is present.

### DETAILED DESCRIPTION

There is presently no injectable device that can, soon after injection, increase the permeability of a peripheral nerve that is completely physiologic and non-toxic to surrounding tissue and to the nerve itself. The present disclosure provides methods that are physiologic and biocompatible, as the compositions used herein comprise ingredients such as saline and glycerol. Moreover, the compositions used in the methods disclosed herein are injectable, thus providing for compositions that infiltrate the target area so that exact precision as to the location of injection and the targeted nerve is not required, and instead injection in the vicinity of the target nerve is efficacious. The methods disclosed herein do not damage surrounding tissue and are neural selective. Further the methods disclosed herein do not induce nerve degeneration, but rather selectively open the tight junction of the peripheral nerves and increase the permeability of peripheral nerves. The methods disclosed herein have a very rapid effect in increasing the permeability of the endoneurium and/or perineurium of a peripheral nerve after administration. Finally, the methods disclosed herein are temporary; the permeability of the nerve cell decreases at and beyond day 3 post treatment.

The methods disclosed herein are beneficial because they are minimally invasive, requiring only an injection through a syringe, as disclosed in U.S. application Ser. No. 15/505,039 (“039 application”; Publication No. US2017/0274078), incorporated in its entirety herein. This administration method is easy to perform and leads to the unexpected results noted above and below, mainly that the barrier around peripheral nerves can be temporarily and immediately be made more permeable without damaging the surrounding tissue. This result can be accomplished using a low amount of slurry that will not cause degeneration of the nerve or damage to the surrounding tissue.

In one embodiment, ice slurry, with a composition as described in the '042 application, is injected around the peripheral nerve of a patient that is targeted for treatment. The amount of slurry administered can be in the range of about 1 mL to about 5 mL, between about 5 mL to about 7 mL, between about 7 mL to about 9 mL, between about 9 mL to about 11 mL, between about 11 mL to about 13 mL, between about 13 mL to about 15 mL, and between about 15 mL to about 20 mL. The temperature of the slurry administered can be in the range of about 0° C. to about −15° C. Within several minutes to hours after the slurry injection, the blood-nerve-barrier has become more permeable and the patient receives a therapeutic drug, compound, or biologic, either by direct injection to the site of target nerve or via systemic administration, e.g., through an IV infusion or through oral intake. The therapeutic drug can also delivered by intravenous injection, local injection, or oral administration.

In one embodiment, the ice slurry will be injected prior to delivery of small molecules and biologics targeting ion channels in the peripheral nervous system (PNS) to treat pain. The delivery of these types of drugs would benefit from opening the blood-nerve barrier by ice slurry to allow these drugs to reach the site of action on the PNS axons. Recent work has shown that voltage-gated sodium ion channels expressed on peripheral nerve axons, especially NaV1.7, NaV1.8 and NaV1.9 to be critical in pain signaling and transmission. Certain peptides, such as the tarantula-based toxin ProTx-II, are known to block specific sodium channels, preventing nerve cells from transmitting signals triggering pain. Biologics, peptides and monoclonal antibodies targeting these channels are being developed for treatment of pain. However, without disrupting the blood-nerve barrier (BNB) these new drugs cannot be delivered to the target site on a peripheral nerve to have an effect due to the BNB. Therefore, the use of ice slurry to safely and temporarily open the BNB for delivery of such drugs will be beneficial for treating any pain caused by blockage of sodium channels on peripheral nerves.

In another embodiment ice slurry treatment precedes local or regional anesthesia, analgesia, and nerve block to peripheral nerves for the reduction of pain from surgeries, neuropathies, or pain syndromes stemming from the PNS. Disrupting the perineurial and endoneurial barriers which impede the delivery of drugs or compounds will be beneficial for reducing the dose of therapeutic compound required, which can limit adverse side effects. Disrupting the perineurial and endoneurial barriers can also extend the duration of the therapeutic effect of drugs by targeted deliver to the site of action. The effectiveness of the drug can also be increased by targeted delivery across BNB. For example, drugs such as tetradotoxin or long-lasting bupivacaine or QX314 are toxic to the body at doses needed to control peripheral nerve pain or induce long lasting anesthesia. Lower systemic doses or local injection permitted by the disruption of the BNB will allow for the safe use of these drugs.

In one aspect the ice slurry is injected prior to local delivery of growth factors that can promote nerve growth or regeneration in inherited or inflammatory neuropathies or after nerve trauma. Following peripheral nerve injury there is a need to promote timely and painless regeneration. In this case, delivery of growth factors such as nerve growth factor (NGF) or brain-derived neurotrophic factor (BNDF) or glial-derived neurotrophic factor (GDNF) will be very beneficial to the patients. Therefore, the use of ice slurry to safely and temporarily open the BNB for delivery of such growth factors or other factors needed for nerve regeneration to injured or traumatized peripheral nerve axons will be very beneficial to patients.

In some embodiments, ice slurry treatment can be used to improve delivery of mRNA or nucleic acid-based therapeutics directly into the PNS for purpose of gene therapy or nerve repair or treatment of nerve diseases such as neuroautoimmune disease, neuropathy, or neuroinflammatory disease. The use of ice-slurry to open BNB will allow more efficient and effective delivery of nucleic acid (DNA or RNA) based therapeutics to the nerve and make targeted gene therapy easier to achieve.

In another aspect ice slurry pretreatment can facilitate targeted delivery of drugs incorporated into cargo with tunable release kinetics. For example, for the delivery of liposomal bupivicaine or other liposomal, extended drugs release across the BNB can potentially extend the duration of their effect in reducing pain, and reduce the amount needed for therapeutic effects further minimizing side effects and increasing the effectiveness by selective and targeted delivery to the site of action.

In one aspect, disclosed herein is a method of administering a drug to a peripheral nerve in a patient, the method comprising injecting an ice slurry in an area around a peripheral nerve in the patient, wherein the injecting increases the permeability of the blood nerve barrier around the peripheral nerve; and administering the drug to the patient.

In another aspect, disclosed herein is a method of administering a substance to a peripheral nerve in a patient, the method comprising injecting an ice slurry in an area around a peripheral nerve in the patient, wherein the injecting increases the permeability of the blood nerve barrier around the peripheral nerve, and administering the substance to the patient.

In another aspect, disclosed herein is a method of administering a drug to a peripheral nerve in a patient, the method comprising injecting an ice slurry in an area around a peripheral nerve in the subject, wherein the injecting increases the permeability of the endoneurial barrier around the peripheral nerve, and administering the drug to the patient.

In another aspect, disclosed herein is a method of administering a drug to a peripheral nerve in a patient, the method comprising injecting an ice slurry in an area around a peripheral nerve in the subject, wherein the injecting increases the permeability of the perineurial barrier around the peripheral nerve, and administering the drug to the patient.

In some embodiments, the administering comprises introducing the drug intravenously, intramuscularly, or orally to the patient. In some embodiments, the administering comprises introducing the drug intravenously to the patient. In some embodiments, the administering comprises introducing the drug intramuscularly to the patient. In some embodiments, the administering comprises introducing the drug orally to the patient.

In some embodiments, the administering comprises injecting the drug into or adjacent to the same location where the ice slurry is injected.

In some embodiments, the administering comprises including the drug in the ice slurry that is administered to the patient.

In some embodiments, the administering comprises administering the drug after injecting the ice slurry.

In some embodiments, the administering occurs 5 minutes after injecting the ice slurry.

In some embodiments, the administering occurs 24 hours after injecting the ice slurry.

In some embodiments, the injecting does not damage the tissues surrounding the peripheral nerve.

In some embodiments, the amount of injected ice slurry is 15 mL.

In some embodiments, the amount of injected ice slurry is 10 mL.

In some embodiments, the amount of injected ice slurry is 5 mL.

In some embodiments, the amount of injected ice slurry is less than 5 mL.

In some embodiments, the substance is selected from the group consisting of a drug, a biologic, nucleic acid, a growth factor, and an anesthetic.

In some embodiments, the administering comprises injecting the drug into the same location where the ice slurry is injected.

### Example 1: Ice Slurry Treatment in Rats and Vascular Permeability Assay

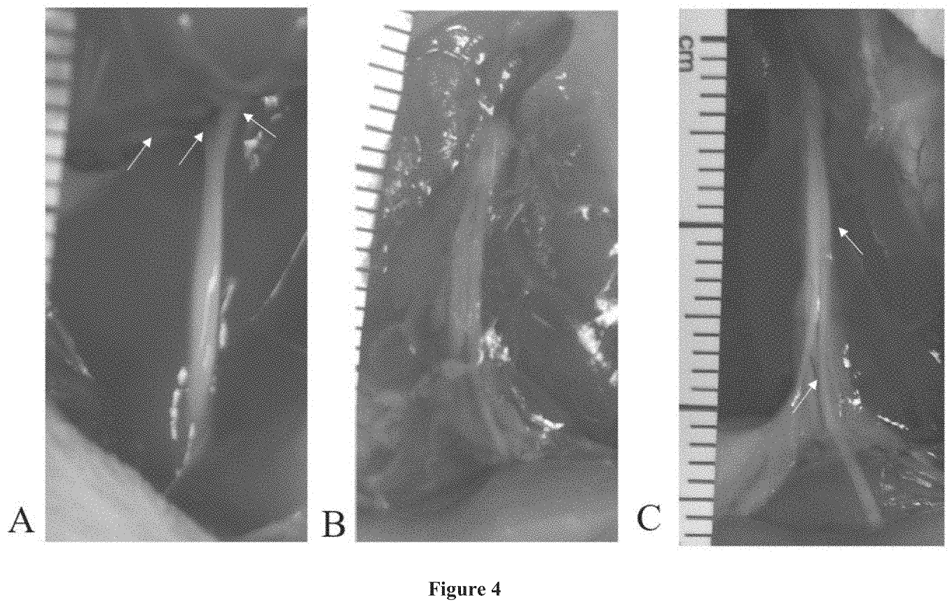

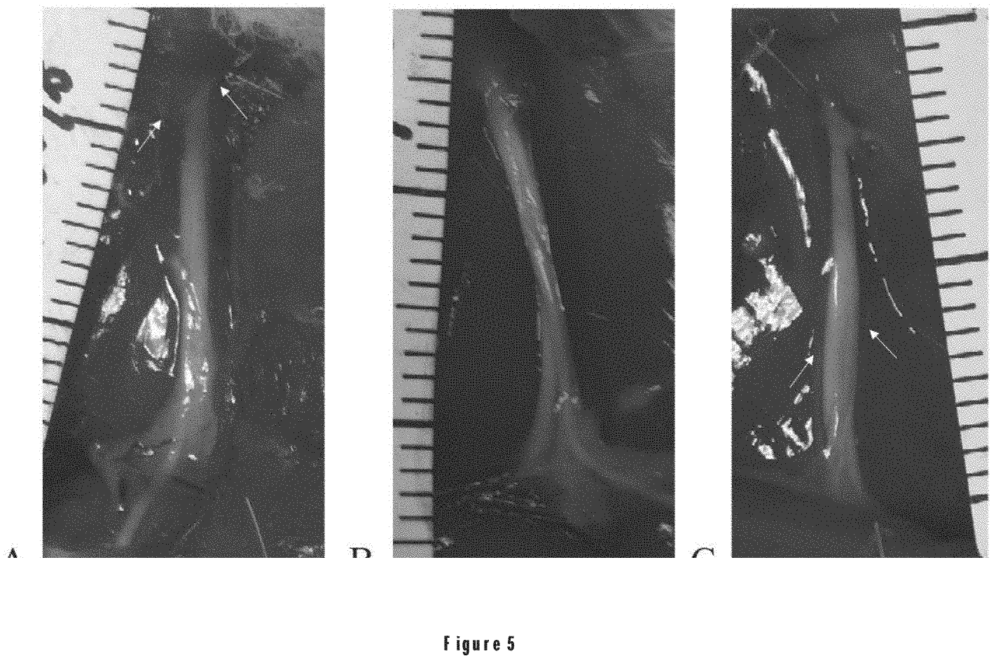

In one example, experiments in 36 rats (n=5 or 6 per group) demonstrate that injection of ice slurry changes the blood-nerve barrier as shown by nerve vascular permeability assays with EB dye at day 1 and day 3 (see FIGS. 4-5). This experiment demonstrated the permeability of the endoneurium of the nerve cells after slurry administration.

Ice slurry treatment was provided as follows. 15 ml of ice slurry at around −5° C. to −6° C. (0.9% sodium chloride with 10% glycerol) or 15 ml of room temperature slurry ((0.9% sodium chloride with 10% glycerol) was injected around the right side sciatic nerve of each animal, under brief anesthesia with inhalational isoflurane (1 to 3% with 1 to 1.5 l/minute oxygen), using standard method of injection. A 15-gauge hypodermic needle was used for the injections. As the control the left side sciatic nerve was left untreated. EB dye (2%, 0.8 ml) was injected through the lateral vein on day 1 or day 3 ice slurry post-treatment. For histologic and imaging data, nerve samples were collected from the rats. One hour after EB dye injection, both the left side sciatic nerve and the right side sciatic nerve were harvested, and images of the sciatic nerves were captured to evaluate levels of blue stain within the sciatic nerves. EB dye was also extracted from half of the harvested tissue samples with formamide overnight. Colorimetric measurements were made at the absorption maximum for EB dye (630 nm) of extravasation. The optical density was converted into a concentration using a standard curve of EB dye in formamide.

As shown in FIGS. 4A-C, at day 1 after injection of 15 ml of ice slurry around the right side sciatic nerve of each animal, the fluorescence tracer (EB dye) was injected through the vein to test the permeability changes of blood vessels in the endoneurium of the sciatic nerve. The sciatic nerves were harvested at 1-hour post EB dye injection. As shown in FIGS. 5A-C, 3 days after injection of 15 ml of ice slurry around the right side sciatic nerve of each animal, EB dye was injected through the vein to test the permeability changes of blood vessels in the endoneurium of the sciatic nerve at 1 hour post EB dye injection. The data presented in FIGS. 4-5 show qualitatively, by the appearance of more gray (as indicated by the arrows) in the tissue surrounding the nerve that there is more EB dye extravasation in ice slurry treated tissue (FIG. 4B) vs. control (FIG. 4A) or room temperature slurry treated tissue (FIG. 4C), thus indicating increased vascular permeability in the treated side. In FIGS. 4A and 4C, there is a gray color (see arrows) that runs inside of the blood vessels where the sciatic nerve goes into the adjacent tissue. However, in FIG. 4B, there is a diffuse gray color distributed to the sciatic nerve and surrounding tissues. The permeability is higher at day 1 post treatment (FIG. 4B) but still present at day 3 (FIG. 5B). Specifically, in FIG. 5A, there is a gray color (see arrows) that runs along the blood vessels in the upper portion of the image where the sciatic nerve goes into the adjacent tissue. In FIG. 5C, there is a slight gray color (see arrows) in the blood vessels around the sciatic nerve. However, in FIG. 5B, there is a diffuse gray color across the entire image, though not as intense when compared to FIG. 4B. Because EB dye has a high affinity for serum albumin, these data demonstrate that ice slurry provides for permeability of molecules at least as large as approximately 66.5 kDa, a size greater than the light chain of IgG. Accordingly, these methods provide for the delivery of not only small molecules, but also protein-based therapeutics.

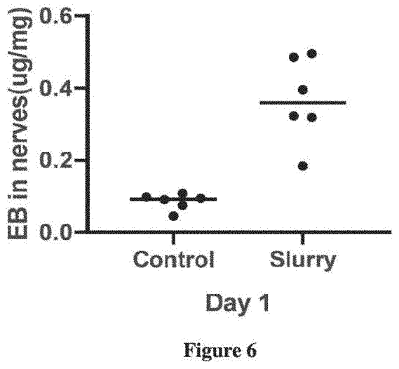

FIG. 6 shows the results of the EB dye extracted from the sciatic nerve samples (n=6 for each group) at day 1 after ice slurry injection. Table 1 below shows analysis of the data presented in FIG. 6. These results demonstrate that much more EB dye is able to permeate the endoneurial barriers at day 1 post ice slurry injection.

TABLE 1

| Table Analyzed | Day 1 Paired t test data |
|---|---|
| Column B | Treated |
| vs. | vs. |
| Column A | Control |
| Paired t test | |
| P value | 0.0011 |
| P value summary** | |
| Significantly different (P < 0.05)? | Yes |
| One- or two-tailed P value? | Two-tailed |
| t, df | t = 6.710, df = 5 |
| Number of pairs | 6 |
| How big is the difference? | |
| Mean of differences (B − A) | 0.2815 |
| SD of differences | 0.1028 |
| SEM of differences | 0.04195 |
| 95% confidence interval | 0.1737 to 0.3893 |
| R squared (partial eta squared) | 0.9001 |
| How effective was the pairing? | |
| Correlation coefficient (r) | 0.7188 |
| P value (one tailed) | 0.0537 |
| P value summary | ns |
| Was the pairing significantly effective? | No |

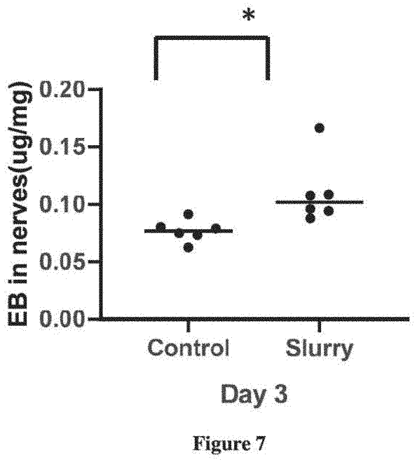

FIG. 7 shows the results of the EB dye extracted from the sciatic nerve samples (n=6 for each group) at day 3 after ice slurry injection. Table 2 below shows analysis of the data presented in FIG. 7. These results demonstrate that more EB dye is able to permeate the endoneurial barriers at day 3 post ice slurry injection, but that the permeability of the BNB is decreasing at day 3.

TABLE 2

| Table Analyzed | Day 3 Paired t test data |
|---|---|
| Column B | Treated |
| vs. | vs. |
| Column A | Control |
| Paired t test | |
| P value | 0.0185 |
| P value summary* | |
| Significantly different (P < 0.05)? | Yes |
| One- or two-tailed P value? | Two-tailed |
| t, df | t = 3.438, df = 5 |
| Number of pairs | 6 |
| How big is the difference? | |
| Mean of differences (B − A) | 0.03326 |
| SD of differences | 0.02370 |
| SEM of differences | 0.009676 |
| 95% confidence interval | 0.008390 to 0.05814 |
| R squared (partial eta squared) | 0.7027 |
| How effective was the pairing? | |
| Correlation coefficient (r) | 0.6489 |
| P value (one tailed) | 0.0816 |
| P value summary | ns |
| Was the pairing significantly effective? | No |

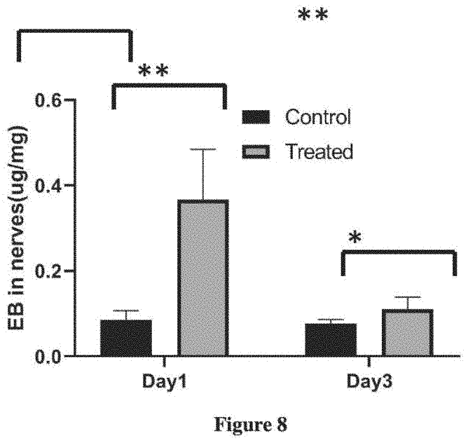

FIG. 8 shows the results of the EB dye extracted from the sciatic nerve samples (n=6 for each group) at day 1 and day 3 after ice slurry injection. Table 3 below shows analysis of the data presented in FIG. 8. These results demonstrate that much more EB dye is able to permeate the endoneurial barriers at day 1 post ice slurry injection, and the amount of EB dye penetration decreases at day 3 post ice slurry injection.

TABLE 3

| Table Analyzed | | Two-way ANOVA-2 Slurry | | | |
|---|---|---|---|---|---|
| Two-way ANOVA | | Ordinary | | | |
| Alpha | 0.05 | | | | |
| Source of Variation | Significant? | % of total variation | | P value | P value summary |
| Interaction | 21.89 | <0.0001 | **** | Yes | |
| Row Factor | 24.88 | <0.0001 | **** | Yes | |
| Column Factor | | 35.19 | <0.0001 | **** | Yes |
| ANOVA table | SS | DF | MS | F (DFn, DFd) | P value |
| Interaction | 0.09243 | 1 | 0.09243 | F (1, 20) = | |
| | | | | 24.27 | |
| | P < 0.0001 | | | | |
| Row Factor | 0.1051 | 1 | 0.1051 | F (1, 20) = | |
| | | | | 27.60 | |
| | P < 0.0001 | | | | |
| Column Factor | | 0.1486 | 1 | 0.1486 | F (1, 20) = |
| | | | | | 39.03 |
| | P < 0.0001 | | | | |
| Residual | 0.07616 | 20 | 0.003808 | | |

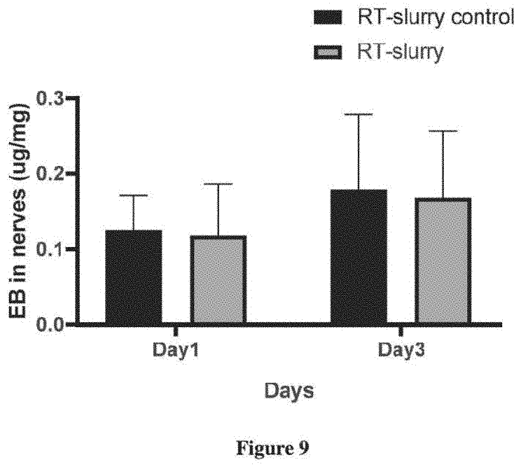

FIG. 9 shows the results of the EB dye extracted from the sciatic nerve samples (n=5) at day 1 and day 3 after room temperature slurry and ice slurry injection. As shown in FIG. 8, when rats were treated with room temperature slurry there was no change in vascular permeability at day 1 or day 3 post treatment. Table 4 below shows analysis of the data presented in FIG. 9.

TABLE 4

| Table Analyzed | RT-slurry | | | | |
|---|---|---|---|---|---|
| Two-way ANOVA | | Ordinary | | | |
| Alpha | 0.05 | | | | |
| Source of Variation | Significant? | % of total variation | | P value | P value summary |
| Interaction | 0.01923 | 0.9535 | ns | No | |
| Row Factor | 12.00 | 0.1581 | ns | No | |
| Column Factor | | 0.3883 | 0.7987 | ns | No |
| ANOVA table | SS (Type III) | DF | MS | F (DFn, DFd) | P value |
| Interaction | 2.180e−005 | 1 | 2.160e−005 | F (1, 16) = | |
| | | | | 0.003512 | |
| | P = 0.9535 | | | | |
| Row Factor | 0.01348 | 1 | 0.01348 | F (1, 16) = | |
| | | | | 2.192 | |
| | P = 0.1581 | | | | |
| Column Factor | | 0.0004137 | 1 | 0.0004137 | F (1, 16) = |
| | | | | | 0.08727 |
| | P = 0.7987 | | | | |
| Residual | 0.09840 | 16 | 0.006150 | | |

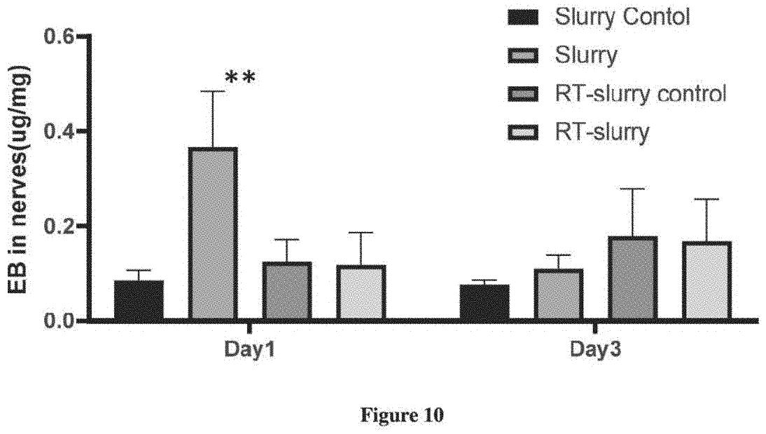

FIG. 10 shows all the combined data of slurry and room temperature treated rats and control (untreated rats designed as slurry control and RT-slurry control in the figure). FIG. 9 shows the results of the EB dye extracted from the sciatic nerve samples (n=6) at day 1 and day 3 after ice slurry or room temperature slurry injection. Slurry control is untreated nerve of the same rat. This table demonstrates that the permeability of the endoneurial barriers has increased at day 1 post ice slurry injection.

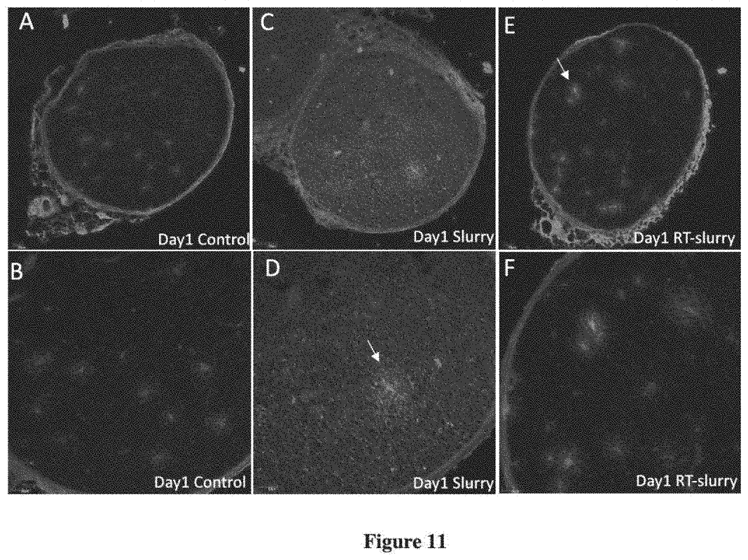

FIGS. 11A-F show confocal images of the EB dye extravasation from the blood vessels within the endoneurium at day 1 post treatment. FIGS. 11A-F show the changes in the permeability of EB dye in the rat sciatic nerve at day 1 after ice slurry injection or room temperature slurry injection. EB dye is shown as the light gray sections on the images, which are where the dye would have a bright red fluorescence. In the control sciatic nerve (FIGS. 11A-B) and room temperature slurry-treated groups (FIGS. 11E-F), the fluorescence was confined to the lumen of the blood vessels (white arrows) and none appeared outside the vascular walls in the endoneurium. FIGS. 11C-D show the dye present in the endoneurium of the treated sciatic nerve, which would be indicated by a bright red fluorescence.

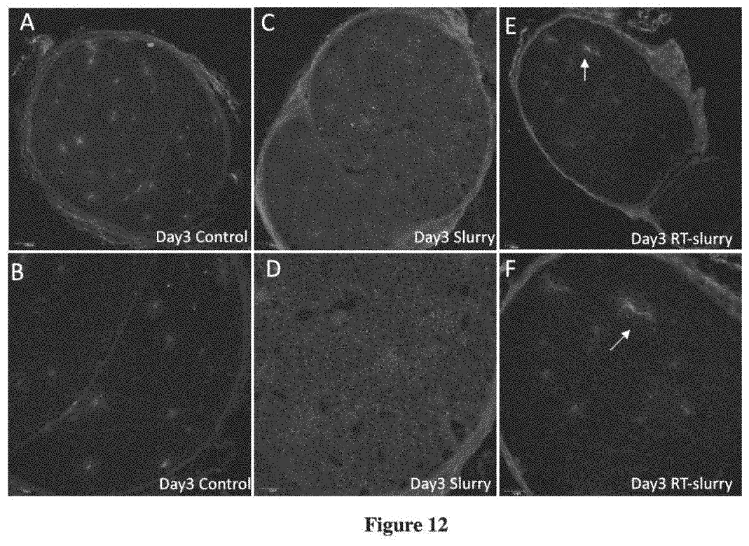

FIGS. 12A-F show confocal images of the EB dye extravasation from the blood vessels within the endoneurium at day 3 post treatment. FIGS. 12A-F show changes in the permeability of EB dye in the rat sciatic nerve at day 3 after ice slurry injection. EB dye is shown as the light gray sections on the images, which are where the dye would have a bright red fluorescence. In the control group (FIGS. 12A-B) and room temperature slurry treated groups (FIGS. 12E-F), the fluorescence was confined to the lumen of the blood vessels (white arrows) and none appeared outside the vascular walls in the endoneurium. FIGS. 12C-D show a distribution of light gray areas in the endoneurium of the treated sciatic nerve, which would be shown as areas of bright red fluorescence in the color images. The data presented in FIGS. 11-12 show that control and room temperature slurry-treated rats do not show any increase in permeability, in contrast to those treated with ice slurry.

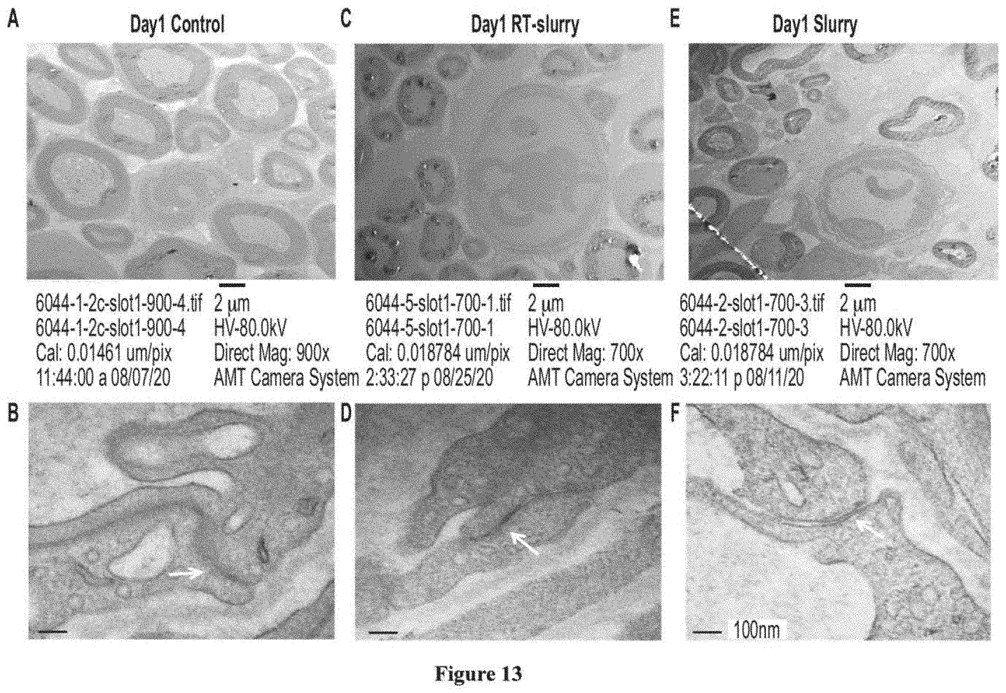

FIGS. 13A-F show TEM images of tight junctions (shown by the white arrows) of the blood vessels at day 1 post treatment. FIGS. 13E-F show an opening of the tight junctions at day 1 post ice slurry injection compared to room temperature slurry injection (FIGS. 13C-D). In the control group (FIGS. 13A-B) and room temperature slurry treated groups (FIGS. 13C-D), the tight junctions are intact. These data show that ice slurry treatment can open tight junctions within one day of injection. There is a notable lack of visible barrier degeneration. These findings indicate unexpected results and are unlikely to be due to nerve degeneration or damage (e.g. Wallerian degeneration), which has an onset of several days post treatment.

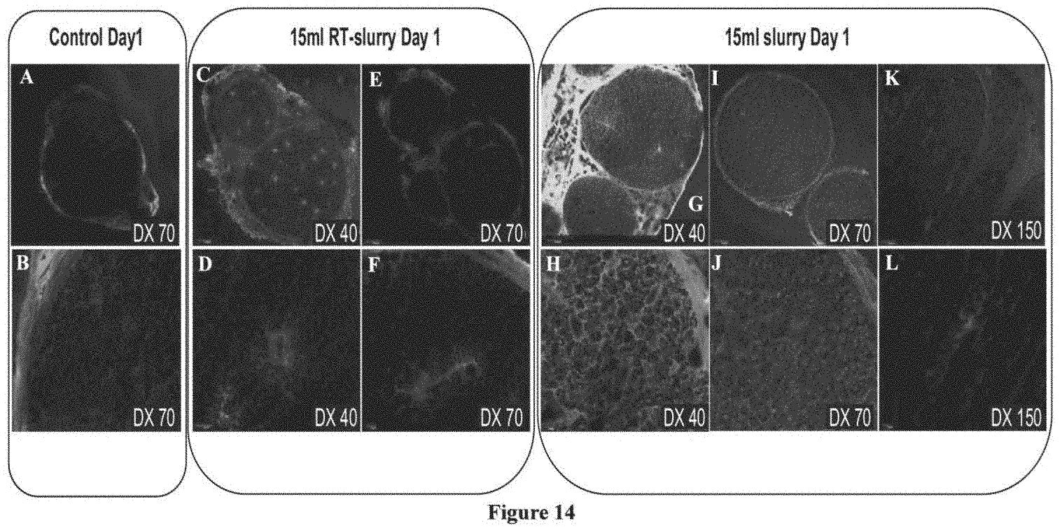

FIGS. 14A-L show confocal images of sciatic nerve on day 1 post slurry treatment and intravenous injection of different sizes of FITC-Dextran (DX) dye.

Ice slurry treatment was provided as in FIGS. 4A-C. 15 ml of ice slurry at around −3.5° C. to −5° C. (0.9% sodium chloride with 10% glycerol) or 15 ml of room temperature slurry ((0.9% sodium chloride with 10% glycerol) was injected around the right side sciatic nerve of each animal, using standard method of injection. As the control, the left side sciatic nerve was left untreated. FITC-Dextran dye of different sizes (40 kDa, 70 kDa, and 150 kDa) were injected through the lateral vein on day 1 post slurry treatment. One hour after injection of the dyes, both the left side sciatic nerve and the right side sciatic nerve were harvested for immunofluorescence confocal imaging. Sciatic nerve samples were processed and embedded in paraffin, and sectioned at 5 μm. Deparaffinized sections were permeabilized with 0.1% Triton X-100, blocked with a solution of goat serum and bovine serum albumin, and treated overnight at 4° C. with primary anti-rat antibodies. Fluorescent secondary antibodies were then applied for 2 hours at room temperature. Confocal images were obtained with an Olympus Fluoview FV1000 (Olympus, USA) laser scanning confocal microscope with IX81 inverted microscope. FIGS. 14G-L show the different permeability levels of the differently sized dyes in the rat sciatic nerve at day 1 after ice slurry injection. The dye is indicated by the lighter areas of the images, which would show a bright green fluorescence in a color image. FIGS. 14G-H show the permeability of DX 40 dye; FIGS. 14I-J show the permeability of DX 70 dye; FIGS. 14K-L show the permeability of DX 150 dye. After 1 day of ice slurry treatment, the nerve was permeable to the particles with a molecular size of 40 kDa and 70 kDa, but was not permeable to particles with a 150 kDa size. In the control group (FIGS. 14A-B) and room temperature slurry treated groups (FIGS. 14C-F), the fluorescence was confined to the lumen of the blood vessels and none appeared outside the vascular walls in the endoneurium.

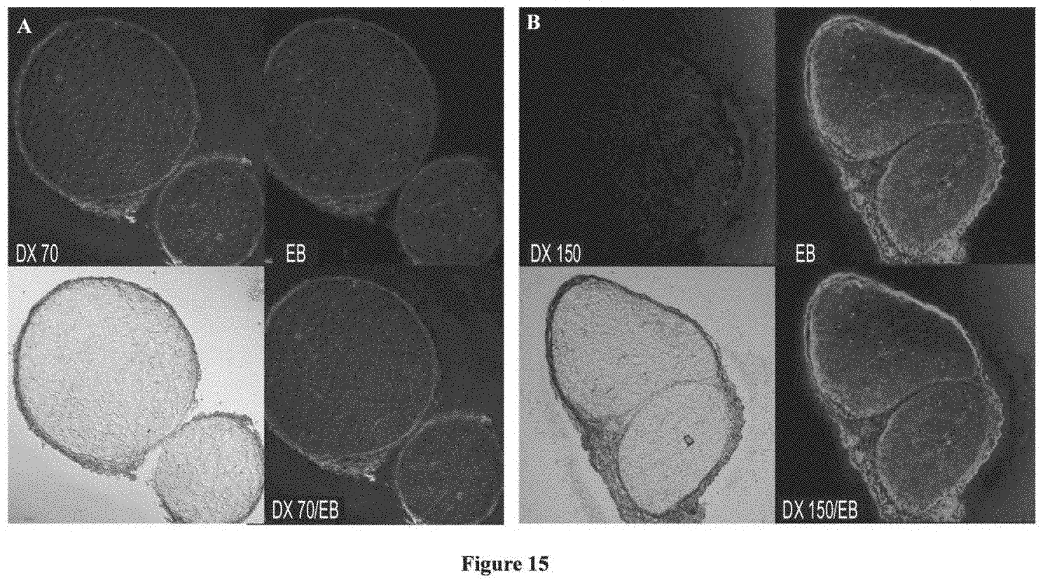

FIGS. 15A-B show confocal and TEM images of the sciatic nerve on day 1 post ice slurry treatment and intravenous injection of different sizes of FITC-Dextran dye (DX 70 and DX 150) and EB dye.

As shown in FIGS. 14A-C, at day 1 after injection of 15 ml of ice slurry around the sciatic nerve, the fluorescent tracers (DX 70, DX 150, and EB) were injected intravenously. The DX 70 dye and EB dye (FIG. 15A) demonstrated similar permeability into the BNB for the ice slurry treated group, as indicated by the similar light portions in those two images. However, the DX 150 dye could not permeate into the endoneurium of the nerve, unlike the EB dye (FIG. 15B). These experiments show that the permeability of the endoneurial barrier induced by ice slurry is size limited.

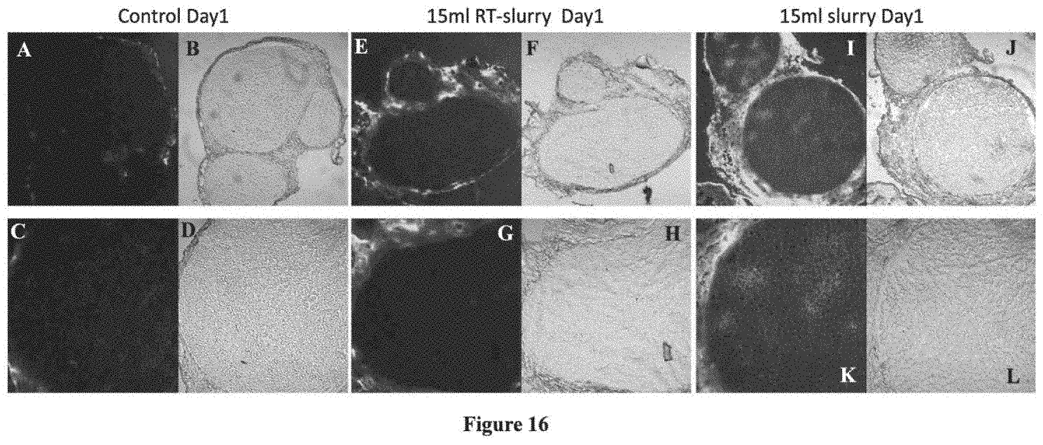

FIGS. 16A-L shows an evaluation of immediate perineurial barrier changes after slurry treatment. 15 ml of ice slurry or room temperature slurry was injected around the right side sciatic nerve of each animal, using standard method of injection. As the control, the left side sciatic nerve was left untreated. DX 70 dye was locally injected five minutes post slurry injection. One day after the DX 70 dye injection, both the left side sciatic nerve and the right side sciatic nerve were harvested for immunofluorescence confocal imaging to evaluate permeability of the perineurium to the dye. Locally injected dye immediately following ice slurry treatment crossed the perineurial barrier (FIGS. 16I-L), which is shown by the lighter gray sections on FIGS. 161 and 16K. Locally injected dye immediately following room temperature slurry treatment or in the control group did not cross the perineurial barrier (FIGS. 16A-H). These experiments show that local injection of ice slurry can immediately render the perineurial barrier permeable. The immediate permeability induced by ice slurry treatment will also be beneficial when applying other treatment, which can immediately follow ice slurry treatment. Also, this shows that the perineurial barrier is made more permeable, as well as the endoneurial barrier.

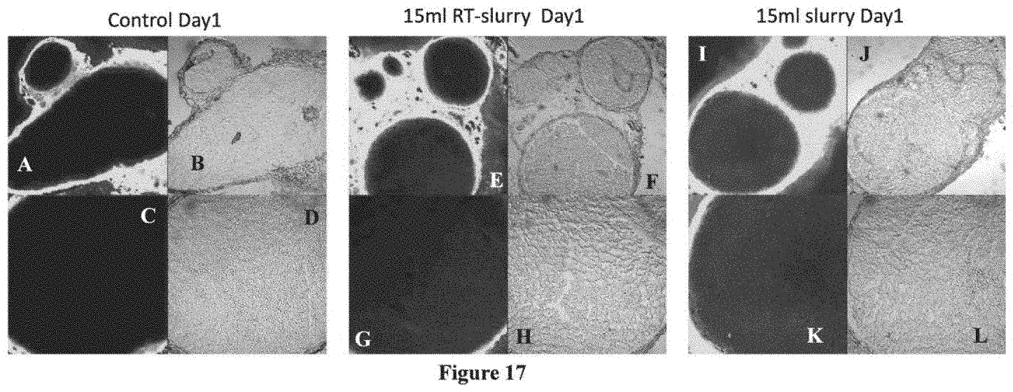

FIGS. 17A-L show confocal and TEM images of the perineurial barrier changes after slurry treatment. 15 ml of ice slurry or room temperature slurry was injected around the right side sciatic nerve of each animal, using standard method of injection. As the control, the left side sciatic nerve was left untreated. DX 70 dye was locally injected one day post slurry injection. One hour after DX 70 dye injection, both the left side sciatic nerve and the right side sciatic nerve were harvested for immunofluorescence confocal imaging to evaluate permeability of the perineurium to the dye. Dye injected locally one day following ice slurry treatment crossed the perineurial barrier (FIGS. 17I-L), which is shown by the lighter portions of FIG. 171 and FIG. 17K. Locally injected dye following room temperature slurry treatment or in the control group did not cross the perineurial barrier (FIGS. 17A-H). These experiments show that local injection of ice slurry can render the perineurial barrier permeable. This also shows that the perineurial barrier is made more permeable, as well as the endoneurial barrier, following injection of ice slurry.

### Example 2: Ice Slurry Treatment in Rats and Evaluation of Perineurium Barrier Breakdown

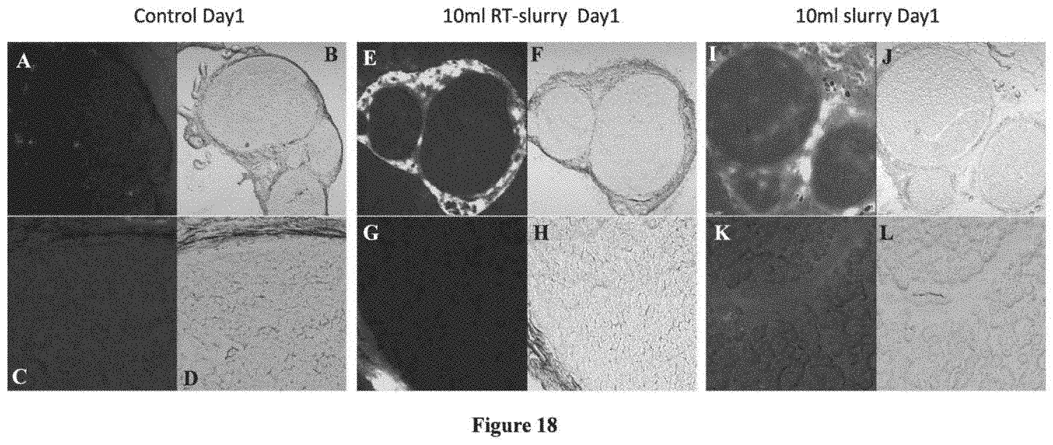

FIGS. 18A-L shows confocal and TEM images of a treated sciatic nerve on day 1 post slurry treatment. 10 ml of ice slurry or 10 ml of room temperature slurry was injected around the right side sciatic nerve of each animal, using a standard method of injection. As the control, the left side sciatic nerve was left untreated. DX 70 dye was locally injected one day post slurry injection. DX 70 dye was locally injected five minutes post slurry injection. One day after the DX 70 dye injection, both the left side sciatic nerve and the right side sciatic nerve were harvested for immunofluorescence confocal imaging to evaluate permeability of the perineurium to the dye. Dye that was locally injected 5 minutes after the ice slurry treatment was shown to cross the perineurial barrier (FIGS. 18I-L). This is indicated by the lighter portions on FIGS. 18I and 18K. Dye that was injected locally 5 minutes after injection of the room temperature slurry treatment or in the control group did not cross the perineurial barrier (FIGS. 18A-H). These experiments show that a smaller volume of ice slurry injected locally can render permeable the perineurial barrier.

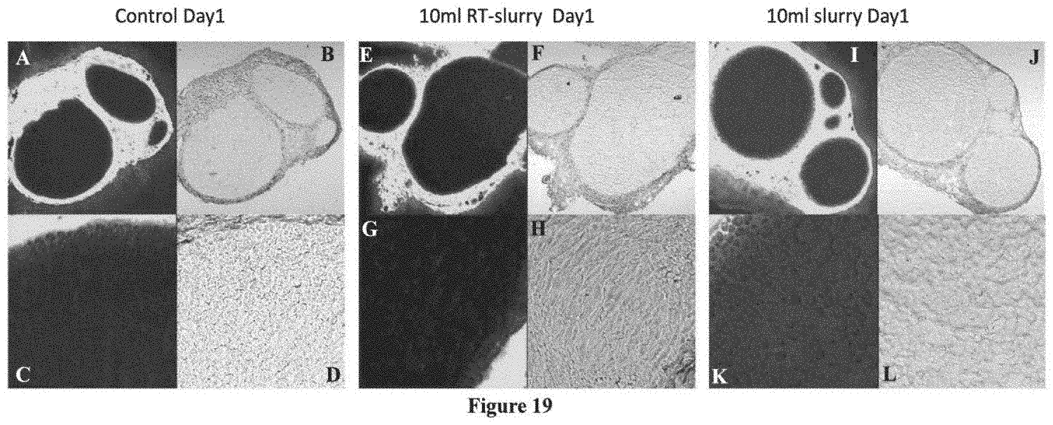

FIGS. 19A-L shows confocal and TEM images of sciatic nerve on day 1 post slurry treatment. 10 ml of ice slurry or 10 ml of room temperature slurry was injected around the right side sciatic nerve of each animal, using standard method of injection. As the control, the left side sciatic nerve was left untreated. DX 70 dye was locally injected one day post slurry injection. One hour after DX 70 dye injection, both the left side sciatic nerve and the right side sciatic nerve were harvested for immunofluorescence confocal imaging to evaluate permeability of the perineurium to the dye. Dye injected locally one day following ice slurry treatment crossed the perineurial barrier (FIGS. 19I-L), which is shown by the lighter gray portions on FIGS. 191 and 19K. Locally injected dye following room temperature slurry treatment or in the control group did not cross the perineurial barrier (FIGS. 19A-H). These experiments show that a smaller volume of ice slurry (10 mL v. 15 mL) injected locally can, either 5 minutes after injection or 1 day after injection, render the perineurial barrier permeable. Further, these findings indicate unexpected results and are unlikely to be due to Wallerian degeneration, which takes several days to begin. The immediate permeability induced by ice slurry treatment is beneficial when applying other treatment, which can be immediately following ice slurry treatment.

### EQUIVALENTS AND SCOPE

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method of administering a drug to a peripheral nerve in a patient, the method comprising:
injecting an ice slurry in an area around a peripheral nerve in the patient, wherein the injecting increases the permeability of the blood nerve barrier around the peripheral nerve; and
administering the drug to the patient.

2. The method of claim 1, wherein the administering comprises introducing the drug intravenously to the patient.

3. The method of claim 1, wherein the administering comprises injecting the drug into or adjacent to the same location where the ice slurry is injected.

4. The method of claim 1, wherein the administering comprises including the drug in the ice slurry that is administered to the patient.

5. The method of claim 1, wherein the administering comprises administering the drug after injecting the ice slurry.

6. The method of claim 5, wherein the administering occurs 5 minutes after injecting the ice slurry.

7. The method of claim 5, wherein the administering occurs 24 hours after injecting the ice slurry.

8. The method of claim 1, wherein the injecting does not damage the tissues surrounding the peripheral nerve.

9. The method of claim 1, wherein the amount of injected ice slurry is 15 mL.

10. The method of claim 1, wherein the amount of injected ice slurry is 10 mL.

11. The method of claim 1, wherein the amount of injected ice slurry is 5 mL.

12. The method of claim 1, wherein the amount of injected ice slurry is less than 5 mL.

13. A method of administering a substance to a peripheral nerve in a patient, the method comprising:
injecting an ice slurry in an area around a peripheral nerve in the patient, wherein the injecting increases the permeability of the blood nerve barrier around the peripheral nerve, and
administering the substance to the patient.

14. The method of claim 13, where the substance is selected from the group consisting of a drug, a biologic, nucleic acid, a growth factor, and an anesthetic.

15. A method of administering a drug to a peripheral nerve in a patient, the method comprising:
injecting an ice slurry in an area around a peripheral nerve in the subject, wherein the injecting increases the permeability of the endoneurial barrier around the peripheral nerve, and
administering the drug to the patient.

16. The method of claim 15, wherein the administering comprises introducing the drug intravenously to the patient.

17. The method of claim 15, wherein the administering comprises injecting the drug into the same location where the ice slurry is injected.

18. The method of claim 15, wherein the administering comprises including the drug in the ice slurry that is administered to the patient.

19. The method of claim 15, wherein the administering comprises administering the drug after injecting the ice slurry.

20. The method of claim 19, wherein the administering occurs 5 minutes after injecting the ice slurry.

21. The method of claim 19, wherein the administering occurs 24 hours after injecting the ice slurry.

22. The method of claim 15, wherein the injecting does not damage the tissues surrounding the peripheral nerve.

23. The method of claim 15, wherein the amount of injected ice slurry is 15 mL.

24. The method of claim 15, wherein the amount of injected ice slurry is 10 mL.

25. A method of administering a drug to a peripheral nerve in a patient, the method comprising:
injecting an ice slurry in an area around a peripheral nerve in the subject, wherein the injecting increases the permeability of the perineurial barrier around the peripheral nerve, and
administering the drug to the patient.

26. The method of claim 1, wherein the administering comprises introducing the drug intravenously, intramuscularly, or orally to the patient.
